(19) United States
(12) Patent Application Publication
(10) Pub. No.: US 2013/0123365 A1
(43) Pub. Date: May 16, 2013

(54) USE OF FTS AND ANALOGS TO TREAT NON-AUTOIMMUNE-ALLERGIC AND NON-ALLERGIC INFLAMMATORY CONDITIONS

(71) Applicants: Yoseph A. Mekori, Kfar Saba (IL); Adam Mor, Tel-Aviv (IL); Yoel Kloog, Herzliya (IL)

(72) Inventors: Yoseph A. Mekori, Kfar Saba (IL); Adam Mor, Tel-Aviv (IL); Yoel Kloog, Herzliya (IL)

(73) Assignee: RAMOT AT TEL-AVIV UNIVERSITY LTD., Tel Aviv (IL)

(21) Appl. No.: 13/809,044

(22) PCT Filed: Jul. 7, 2011
(86) PCT No.: PCT/IL11/00541

§ 371 Date: Jan. 8, 2013

Related U.S. Application Data
(60) Provisional application No. 61/362,478, filed on Jul. 8, 2010.

(51) Int. Cl. A61K 31/60

(52) U.S. Cl. CPC A61K 31/60
USPC 514/568

(57) ABSTRACT

Disclosed are methods of treating a mammalian subject afflicted with a non-autoimmune inflammatory condition, comprising administering to the subject a pharmaceutical composition comprising an effective amount of S-farnesylthiosalicylic acid (FTS) or a structural analog thereof, and compositions useful in the practice of the methods.

### BACKGROUND OF THE INVENTION

Inflammation is a biological response that can result from a noxious stimulus and is normally intended to remove that stimulus or ameliorate its effects. Although normally intended to promote survival, inflammation can cause damage to the host, especially in mammals. The stimulus or insult initiating inflammation can be caused by endogenous factors (e.g., an auto-antigen or irritating body fluid) or exogenous factors (e.g., a foreign body or infectious agent).

Inflammation has been classified as “acute” and “chronic”. Acute inflammation is typically of relatively short duration, lasting minutes to hours and, in some cases, a few days. Acute inflammation can be characterized by the exudation of fluid and plasma proteins and the accumulation of polymorphonuclear leukocytes (PMNs) at the site of the insult. Acute inflammation usually includes an increase in blood flow to the area of the insult mediated by cellular molecules released in response to the insult. Increased vascular permeability also results from cellular mediators and leads to an accumulation of protein-rich fluid. Important mediators of this increased blood flow and vascular permeability include histamine from mast cells, serotonin and bradykinin.

In acute inflammation, PMNs are also attracted to the area of insult and migrate out of the blood stream toward the insult. The PMNs release toxic metabolites and proteinases that can cause tissue damage. These proteinases include proteins in the complement system, which can damage cell membranes and kallikreins which generate bradykinin. Acute inflammation can undergo complete resolution, lead to the formation of an abscess, result in scarring fibrosis or progress to chronic inflammation.

Chronic inflammation is of longer duration, lasting weeks to months, and possibly years, in which tissue destruction and biological processes that are intended to repair the injury are simultaneously ongoing. Chronic inflammation more typically involves lymphocytes and macrophages and may also include a proliferation of blood vessels, fibrosis and/or necrosis. Chronic inflammation can result from a number of conditions including persistent infections, prolonged exposure to toxic agents, and autoimmune reactions. Chronic inflammation is often maintained by the production of cytokines by lymphocytes and macrophages at the site of the persistent insult. Chronic inflammation can result in permanent tissue damage or complete healing.

Hypersensitivity generally refers to inflammation that causes damage to the host, in which the damage outweighs the benefit to the host. Hypersensitivity can result in significant pathology including, e.g., anaphylaxis, transplant rejection, and autoimmune diseases. The most common type of hypersensitivity is allergy.

Independently of the inducing factor (and the length of the exposure), an inflammatory reaction is mediated by a varied number and type of cells and molecules, the later including cytokines, growth factors, clotting factors, enzymes, neurotransmitters and complement proteins, among others. These molecules are primarily secreted by fibroblasts, endothelial and infiltrating cells (e.g., macrophages, lymphocytes, mast cells, polymorphonuclear cells, etc), and local nerves in response to the insulting agent. The mixture and amount of cytokines therein released will depend on the type, concentration and exposure time of the inducing agent. Therefore, these proteins could mediate from an acute local inflammatory reaction to systemic life-threatening responses (e.g., acute systemic inflammatory response syndrome, SIRS; multiple organ failure as in septic shock; anaphylaxis, etc). In chronic inflammatory processes, the cytokines continuously recruit more and more infiltrating cells that generate, for example, granulomas, induration of the tissues, and encapsulated abscesses. In any case, proteins secreted during an inflammatory process are central players in the grade and persistence of the final reaction.

Stimulation of the aforementioned cells by the induction agent leads to a cascade of intracellular signaling events that ultimately result in production and secretion of cytokines and other inflammatory mediators that constitute the pro-inflammatory response. While the pro-inflammatory response is crucial for effective clearance of the pathogen or allergen, the inflammatory mediators also cause tissue damage and inflammation. Hence, a balance needs to be maintained between the activation and down-regulation of this response in order to avoid severe tissue damage (Cohen, Nature 420:885-91 (2002)). Dysregulation of this response could induce local damage (e.g., lung fibrosis) or could lead to potentially lethal conditions like septic shock and systemic inflammatory response syndrome (SIRS). Thus, microbes, allergens, endotoxins and many other molecules induce the production of pro-inflammatory mediator proteins by different cells in the human body. The combined effects of all these molecules in living tissues could mediate changes in the clotting system, wound healing process, anti-microbial activity, antibody production and the perception of pain, among many other reactions.

Mast cells (MC) are tissue elements derived from a particular subset of hematopoietic stem cells that express CD34, c-kit and CD13 antigens (Kirshenbaum, et al., Blood 94:2333-42 (1999) and Ishizaka, et al., Curr Opin. Immunol. 5:937-43 (1993)). Immature MC progenitors circulate in the bloodstream and differentiate in tissues. Mast cells play an important protective role in terms of wound healing and defense against pathogens.

It is now believed that mast cells are implicated in or contribute to the genesis of diseases such as autoimmune diseases, allergic diseases, tumor angiogenesis, inflammatory diseases, polyarthritis, inflammatory bowel diseases (IBD), and interstitial cystitis.

In addition to containing many granules rich in histamine and heparin, mast cells express a high-affinity receptor FcεRI for the Fc region of IgE, the least-abundant member of the antibodies. This receptor is of such high affinity that binding of IgE molecules is essentially irreversible. As a result, mast cells are coated with IgE. IgE is produced by plasma cells which are the antibody-producing cells of the immune system. IgE molecules, like all antibodies, are specific to one particular antigen. It is postulated that upon activation (e.g., in response to direct injury, allergen-IgE binding which leads to cross-linking of IgE receptors, or by activated complement proteins), mast cells participate in the destruction of tissues by releasing its characteristic granules (a process known as degranulation) and a cocktail of different proteases and mediators which aside from the release of histamine as a result of degranulation, include other biogenic amines, proteoglycans, neutral proteases, lipid-derived mediators (prostaglandins (e.g., PGD₂), thromboxanes and leucotrienes), and various cytokines (IL-1, IL-2, IL-3, IL-4, IL-5, IL-6, IL-8, TNF-alpha, GM-CSF, MIP-1a, MIP-1b, MIP-2 and IFN-gamma). These chemicals activate vascular leakage of cells and fluids, broncho-constriction, intestinal hypermotility, inflammation, and tissue remodeling.

Proposed mast cell-targeted therapy for treatment of inflammation has included use of prostaglandin D2 receptor antagonists (e.g., U.S. 2008/0194600), and via inhibition of the anti-apoptotic A-1/bfl-1 gene or expression product (U.S. Pat. No. 6,465,187).

### BRIEF SUMMARY OF THE INVENTION

In a first aspect, the present invention is directed to the compound S-farnesylthiosalicylic acid (FTS) or a structural analog thereof, collectively defined in accordance with formula (I) herein for use in a method of treating a mammalian subject afflicted with a non-autoimmune inflammatory condition.

Another aspect of the present invention is directed to a method of treating a mammalian subject afflicted with a non-autoimmune inflammatory condition, comprising administering to the subject a pharmaceutical composition comprising an effective amount of S-farnesylthiosalicylic acid (FTS) or a structural analog thereof, collectively defined in accordance with formula (I) herein, and a pharmaceutically acceptable carrier.

Without intending to be bound by any particular theory of operation, Applicants believe that FTS and its analogs exert their inhibitory effect on mast cells via the Ras pathway. The working examples demonstrate that FTS inhibited Ras activation in MC; FTS preferentially inhibited prostaglandin PG) D₂ and tumor necrosis factor (TNF)-release without having any significant effect on MC beta-hexosaminidase secretion; and that in vivo, administration of FTS inhibited the late phase of passive cutaneous anaphylaxis reaction, and that the time course of FTS-induced inhibition in vivo correlated with mediators release and not with degranulation.

Accordingly, a related aspect of the present invention is directed to a method of inhibiting activation and/or generation and/or release of proinflammatory mediators from mast cells in vivo, comprising administering to a mammalian subject afflicted with a non-autoimmune inflammatory condition a pharmaceutical composition comprising an effective amount of S-farnesylthiosalicylic acid (FTS) or a structural analog thereof, collectively defined in accordance with formula (I) herein, and a pharmaceutically acceptable carrier.

In some embodiments, the inhibitory effect of FTS and its analogs includes inhibition of prostaglandin D₂ (PG D₂) generation and release of tumor necrosis factor-α.

In a further aspect of the invention is directed to use of S-farnesylthiosalicylic acid (FTS) or a structural analog thereof, collectively defined in accordance with formula (I) herein, for the preparation of a pharmaceutical composition for treating a mammalian subject afflicted with a non-autoimmune inflammatory condition and/or ameliorating at least one symptoms of said condition.

In yet a further aspect of the invention is directed to use of S-farnesylthiosalicylic acid (FTS) or a structural analog thereof, collectively defined in accordance with formula (I) herein, for treating a mammalian subject afflicted with a non-autoimmune inflammatory condition and/or ameliorating at least one symptoms of said condition.

In yet a further aspect, the invention is directed at a pharmaceutical composition comprising S-farnesylthiosalicylic acid (FTS) or a structural analog thereof as an active agent for treating a non-autoimmune inflammatory condition and/or ameliorating at least one symptoms of said condition.

In some embodiments of all aspects of the invention, the subject has an allergic inflammatory condition, and is treated with S-trans, trans-farnesylthiosalicylic acid. The conditions treatable in accordance with the present invention are also non-cancerous.

### BRIEF DESCRIPTION OF THE DRAWINGS

FIGS. 1A-C illustrate that FTS inhibits Ras activation in mast cells. LAD2 cells expressing RFP-N-Ras and GFP tagged Raf-1-RBD were analyzed before and after stimulation by cross-linking the FCεRI and after pretreated with 25 μM of FTS (FIG. 1A). Stimulated LAD2 cells were subjected to Raf-1-RBD pull down assay. Cells were stimulated by cross-linking of the FCεRI, before and after pretreatment with 25 μM of FTS (FIG. 1B). Quantification of the same experiment (n=4, mean (±SEM), FIG. 1C).

FIGS. 2A-C illustrate preferential inhibition of tumor necrosis factor-αrelease. LAD2 cells were activated by cross-linking the FCεRI with and without pretreatment with different concentrations of FTS (as indicted). The levels of ε-hexosaminidase secretion were studied 20 minutes after stimulation (FIG. 2A). PGD₂ levels were studied 6 hours after stimulation (FIG. 2B). TNF-α levels were studied 3 hours after stimulation (FIG. 2C).

FIG. 3 illustrates in vivo inhibition of passive cutaneous anaphylaxis reaction by FTS. Mice were given either oral FTS (100 mg/kg), or vehicle. The left footpads of all mice were injected subcutaneously with IgE (anti-HSP), while the right footpads were injected with diluents. Footpad swelling was measured at different time points after antigen (DNP₃₀₋₄₀-HAS) challenge.

### DETAILED DESCRIPTION

The non-autoimmune inflammatory conditions suitable for treatment by the pharmaceutical compositions and methods in accordance with the present invention include allergic inflammation and non-allergic inflammation. The term “allergic inflammation” refers to the manifestations of immunoglobulin E (IgE)-related immunological responses. (Manual of Allergy and Immunology, Chapter 2, Alvin M. Sanico, Bruce S. Bochner, and Sarbjit S. Saini, Adelman et al, ed., Lippincott, Williams, Wilkins, Philadelphia, Pa., (2002)). Allergic inflammation includes pulmonary inflammatory diseases such as allergic rhinosinusitis, asthma (i.e. a disorder characterized by increased responsiveness of the trachea and bronchi to various stimuli, which results in symptoms that include, but are not limited to, wheezing, cough, shortness of breath, dyspnea, and the like. Asthma includes, for example, allergic, childhood, atopic and occupational asthma), hay fever, allergic rhinitis, exercise-induced bronchoconstriction, allergic pneumonitis, ocular allergic conditions such as allergic conjunctivitis, giant papillary conjunctivitis, vernal conjunctivitis, and atopic keratoconjunctivitis; inflammatory skin conditions such as urticaria (hives), angiodema (wheal and flare), contact dermatitis, atopic eczema (e.g., poison ivy), atopic dermatis, and insect venom allergic reactions (e.g., sting allergy); gastrointestinal inflammatory conditions such as eosinophilic eosinophilic esophagitis; and systemic (multi-system) allergic conditions such as ana-phylactic and anaphylactoid reactions and anaphylaxis and other conditions induced by allergens such as food, drugs, latex and pets.

Non-IgE-mediated allergic conditions that are mast cell-dependent are also treatable with the methods and pharmaceutical compositions in accordance with the present invention, an example of which is sarcoidosis.

Non-autoimmune inflammatory conditions that are also non-allergic in nature, and which are suitable for treatment in accordance with the methods and pharmaceutical compositions of the present invention include adult respiratory distress syndrome (ARDS), musculoskeletal inflammatory conditions such as serum sickness and urticarial vasulitis; and systemic conditions such as mastocytosis and hypereosinophilic syndrome.

The subjects for treatment with the methods and pharmaceutical compositions of the present invention are mammals, including humans and experimental or disease-model mammals, and other non-human mammals including domestic animals.

FTS and its structural analogs useful in the methods, uses and compositions of the present invention may be collectively represented by the formula:

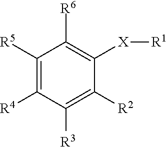

wherein
X represents S;
wherein R1 represents farnesyl or geranyl-geranyl; R² is COOR⁷, CONR⁷R⁸, or COOCHR⁹OR¹⁰, wherein R⁷ and R⁸ are each independently hydrogen, alkyl, or alkenyl, including linear and branched alkyl or alkenyl, which in some embodiments includes C1-C4 alkyl or alkenyl;

wherein R⁹ represents H or alkyl; and

wherein R¹⁰ represents alkyl, including linear and branched alkyl and which in some embodiments represents C1-C4 alkyl; and

wherein R³, R⁴, R⁵ and R⁶ are each independently hydrogen, alkyl, alkenyl, alkoxy (including linear and branched alkyl, alkenyl or alkoxy and which in some embodiments represents C1-C4 alkyl, alkenyl or alkoxy), halo, trifluoromethyl, trifluoromethoxy, or alkylmercapto.

In some embodiments wherein any of R⁷, R⁸, R⁹ and R¹⁰ represents alkyl, it is methyl or ethyl.

Thus, aside from FTS (e.g., the isomer S-trans,trans-farnesylthiosalicylic acid, wherein R¹ is farnesyl, R² is COOR⁷, and R⁷ is hydrogen), in some embodiments, the FTS analog is halogenated, e.g., 5-chloro-FTS (wherein R¹ is farnesyl, R² is COOR⁷, R⁴ is chloro, and R⁷ is hydrogen), and 5-fluoro-FTS (wherein R¹ is farnesyl, R² is COOR⁷, R⁴ is fluoro, and R⁷ is hydrogen).

In other embodiments, the FTS analog is FTS-methyl ester (wherein R¹ represents farnesyl, R² represents COOR⁷, and R⁷ represents methyl).

In yet other embodiments, the Ras antagonist is an alkoxyalkyl S-prenylthiosalicylate or an FTS-alkoxyalkyl ester (wherein R² represents COOCHR⁹OR¹⁰). Representative examples include methoxymethyl S-farnesylthiosalicylate (wherein R¹ is farnesyl, R⁹ is H, and R¹⁰ is methyl); methoxymethyl S-geranylgeranylthiosalicylate (wherein R¹ is geranylgeranyl, R⁹ is H, and R¹⁰ is methyl); methoxymethyl 5-fluoro-S-farnesylthiosalicylate (wherein R¹ is farnesyl, R⁵ is fluoro, R⁹ is H, and R¹⁰ is methyl); and ethoxymethyl S-farnesylthiosalicyate (wherein R¹ is farnesyl, R⁹ is methyl and R¹⁰ is ethyl). In each of the embodiments described above, unless otherwise specifically indicated, each of R³, R⁴, R⁵ and R⁶ represents hydrogen.

In yet other embodiments, the FTS analog is FTS-amide (wherein R¹ represents farnesyl, R² represents CONR⁷R⁸, and R⁷ and R⁸ both represent hydrogen); FTS-methylamide (wherein R¹ represents farnesyl, R² represents CONR⁷R⁸, R⁷ represents hydrogen and R⁸ represents methyl); or FTS-dimethylamide (wherein R¹ represents farnesyl, R² represents CONR⁷R⁸, and R⁷ and R⁸ each represents methyl).

The term “alkyl” refers to a saturated aliphatic hydrocarbon having between 1 and 12 carbon atoms, preferably between 1 and 6 carbon atoms, which may be arranged as a straight chain or branched chain, or as a cyclic group. These are, for example, methyl, ethyl, propyl, isobutyl, and butyl.

The alkyl group may be unsubstituted or substituted with one or more of a variety of groups selected from halogen, hydroxyl, alkyloxy, alkylthio, arylthio, alkoxy, alkylcarbonyl, carbonyl, alkoxycarbonyl, ester, amido, alkylamido, dialkylamido, aryl, benzyl, aryloxy, nitro, amino, alkyl or dialkylamino, carboxyl, thio, and others, each optionally being isotopically labeled. When substituted by a terminal group, the alkyl is an alkylene having between 1 and 12 carbon atoms. When the alkyl or alkylene group contains one or more double bonds it is referred herein as an “alkenyl”.

The term “alkoxy” as used herein refers to the —O—-(alkyl) group, where the point of attachment is through the oxygen-atom and the alkyl group is as defined hereinbefore.

The term “halogen” or “halo” as used herein refers to —Cl, —Br, —F, or —I groups.

The term “ester” as used herein refers to a —C═(O)—O—, where the points of attachment are through both the C-atom and O-atom. One or both oxygen atoms of the ester group can be replaced with a sulfur atom, thereby forming a “thioester”, i.e., a —C═(O)—S—, —C═(S)—O— or —C═(S)—S— group.

### Compositions and Methods

The term “treatment” as used herein refers to the administering of a therapeutic amount of the composition of the present invention which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

The term “effective amount” as used herein, refers to a sufficient amount of an active ingredient as represented by formula (I) that will ameliorate at least one symptom or underlying biochemical manifestation of the non-autoimmune inflammatory condition, such as inhibition of release of proinflammatory mediators from mast cells, diminish extent or severity or delay or retard progression, or achieve complete healing and regression of the condition. A representative but non-exhaustive list of symptoms and signs of inflammatory conditions include itching (pruritis), runny nose, blurred vision, edema, pain, coughing, difficulty breathing (e.g., wheezing), fever, sweating (e.g., at night), loss of function, redness, scaling, blistering, hyper- and hypo-pigmentation, hypotension, chest pain, diarrhea, arthralgia and myalgia. Symptoms and signs in connection with the non-allergic inflammatory conditions include headache, weakness, fatigue, loss of vision, oral ulcers, hair loss, swollen joints, back pain, pleuritic chest pain, hematuria, weight loss, and dyspnea. Appropriate “effective” amounts for any subject can be determined using techniques, such as a dose escalation study. Specific dose levels for any particular subject will depend on several factors such as the potency of the active ingredient represented by formula (I), the age, weight, and general health of the subject, and the severity of the disorder. The average daily dose of the active ingredient of formula (I) generally ranges from a minimum of 1 mg/Kg, 2 mg/Kg, 3 mg/Kg, 4 mg/Kg, 5 mg/Kg, 6 mg/Kg, 7 mg/Kg, 8 mg/Kg, 9 mg/Kg, 10 mg/Kg, 12 mg/Kg, 14 mg/Kg to a maximum of 12 mg/Kg, 14 mg/Kg, 16 mg/Kg, 18 mg/Kg, 20 mg/Kg, and 30 mg/Kg [Please confirm]. Thus the daily dose of the active ingredient for an adult may range for example from about 200 mg to about 2000 mg, in some embodiments from about 400 to about 1600 mg, and some other embodiments from about 600 to about 1200 mg, and in yet other embodiments, from about 800 mg to about 1200 mg.

The terms “administer,” “administering”, “administration,” and the like, as used herein, refer to the methods that may be used to enable delivery of the active ingredient to the desired site of biological action. Medically acceptable administration techniques suitable for use in the present invention are known in the art. See, e.g., Goodman and Gilman, The Pharmacological Basis of Therapeutics, current ed.; Pergamon; and Remington's, Pharmaceutical Sciences (current edition), Mack Publishing Co., Easton, Pa. In some embodiments, the active ingredient is administered orally. In other embodiments, the active ingredient is administered parenterally (which for purposes of the present invention, includes intravenous, subcutaneous, intraperitoneal, intramuscular, intravascular and infusion). In yet other embodiments, the active ingredient is administered transdermally (e.g., topically). As used herein, topical administration refers to non-enteral and non-parenteral modes of administration, and thus includes direct or indirect application to the skin, as well as inhalational (e.g., via aerosol) and ocular (e.g., eye drops or eardrops) administration.

The term “pharmaceutical composition”, as used herein, refers to a combination or mixture of the active ingredient and a pharmaceutically acceptable carrier, and optionally, a pharmaceutically acceptable excipient, which as known in the art include substances or ingredients that are non-toxic, physiologically inert and do not adversely interact with the active ingredient of formula (I) (and any other additional active agent(s) that may be present in the composition). Carriers facilitate formulation and/or administration of the active agents.

The term “pharmaceutically acceptable carrier” (which are interchangeably referred to throughout the specification as “carriers”) refers to any vehicle, adjuvant, excipient, diluent, which is known in the field of pharmacology for administration to a human subject and is approved for such administration. The choice of carrier will be determined by the particular active agent, for example, its dissolution in that specific carrier (hydrophilic or hydrophobic), as well as by other criteria such as the mode of administration.

Oral compositions suitable for use in the present invention may be prepared by bringing the active ingredient(s) into association with (e.g., mixing with) the carrier, the selection of which is based on the mode of administration. Carriers are generally solid or liquid. In some cases, compositions may contain solid and liquid carriers. Compositions suitable for oral administration that contain the active are in some embodiments in solid dosage forms such as tablets (e.g., including film-coated, sugar-coated, controlled or sustained release), capsules, e.g., hard gelatin capsules (including controlled or sustained release) and soft gelatin capsules, powders and granules. The compositions, however, may be contained in other carriers that enable administration to a patient in other oral forms, e.g., a liquid or gel. Regardless of the form, the composition is divided into individual or combined doses containing predetermined quantities of the active ingredient.

Oral dosage forms may be prepared by mixing the active ingredient, typically in the form of an active pharmaceutical ingredient with one or more appropriate carriers (optionally with one or more other pharmaceutically acceptable excipients), and then formulating the composition into the desired dosage form e.g., compressing the composition into a tablet or filling the composition into a capsule (e.g., a hard or soft gelatin capsule) or a pouch. Typical carriers and excipients include bulking agents or diluents, binders, buffers or pH adjusting agents, disintegrants (including crosslinked and super disintegrants such as croscarmellose), glidants, and/or lubricants, including lactose, starch, mannitol, microcrystalline cellulose, ethylcellulose, sodium carboxymethylcellulose, hydroxypropylmethylcellulose, dibasic calcium phosphate, acacia, gelatin, stearic acid, magnesium stearate, corn oil, vegetable oils, and polyethylene glycols. Coating agents such as sugar, shellac, and synthetic polymers may be employed, as well as colorants and preservatives. See, Remington's Pharmaceutical Sciences, The Science and Practice of Pharmacy, 20th Edition, (2000).

Liquid form compositions include, for example, solutions, suspensions, emulsions, syrups, elixirs and pressurized compositions. The active agent(s), for example, can be dissolved or suspended in a pharmaceutically acceptable liquid carrier such as water, an organic solvent (and mixtures thereof), and/or pharmaceutically acceptable oils or fats. Examples of liquid carriers for oral administration include water (particularly containing additives as above, e.g., cellulose derivatives, according to some embodiments—in suspension in sodium carboxymethyl cellulose solution), alcohols (including monohydric alcohols (including monohydric alcohols and polyhydric alcohols, e.g., glycerin and non-toxic glycols) and their derivatives, and oils (e.g., fractionated coconut oil and arachis oil). The liquid composition can contain other suitable pharmaceutical excipients such as solubilizers, emulsifiers, buffers, preservatives, sweeteners, flavoring agents, suspending agents, thickening agents, colorants, viscosity regulators, stabilizers and osmoregulators.

Carriers suitable for preparation of compositions for parenteral administration include Sterile Water for Injection, Bacteriostatic Water for Injection, Sodium Chloride Injection (0.45%, 0.9%), Dextrose Injection (2.5%, 5%, 10%), Lactated Ringer's Injection, and the like. Dispersions can also be prepared in glycerol, liquid polyethylene glycols and mixtures thereof, and in oils. Compositions may also contain tonicity agents (e.g., sodium chloride and mannitol), antioxidants (e.g., sodium bisulfite, sodium metabisulfite and ascorbic acid) and preservatives (e.g., benzyl alcohol, methyl paraben, propyl paraben and combinations of methyl and propyl parabens).

Transdermal (e.g., topical) compositions may take a variety of forms such as gels, creams, lotions, aerosols and emulsions. Representative carriers thus include lubricants, wetting agents, emulsifying and suspending agents, preservatives, anti-irritants, emulsion stabilizers, film formers, gel formers, odor masking agents, resins, hydrocolloids, solvents, solubilizers, neutralizing agents, permeation accelerators, pigments, quaternary ammonium compounds, refatting and superfatting agents, ointment, cream or oil base materials, silicone derivatives, stabilizers, sterilizing agents, propellants, drying agents, opacifiers, thickeners, waxes, emollients, and white oils In addition, the topical preparations of the present invention can be applied and then covered with a bandage, or patch, or some other occlusive barrier, or may be provided as part of a pre-made, ready-to-use topical device, such as a bandage, pad, patch (e.g., transdermal patch of the matrix or reservoir type) or the like. Thus, the composition containing the active ingredient of formula (I) may be applied to a gauze, pad, swab, cotton ball, batting, bandage, patch or occlusive barrier, or in a well or reservoir or as part of a unitary adhesive or nonadhesive mixture, or sandwiched between a peelable or removable layer and a backing layer, which often forms the reservoir, which is occlusive.

Carriers for aerosol formulation, in which the active may be present in finely divided or micronized form, include lactose and propellants such as hydrocarbons (HCF) (propane and n-butane), ether-based propellants such as dimethyl ether and methyl ethyl ether, and hydrofluoroalkanes (HFC) such as HFA 134a and HFA 227. Excipients may also be present, e.g., for such purposes as to improve drug delivery, shelf life and patient acceptance. Examples of excipients include wetting agents (e.g., surfactants), dispersing agents, coloring agents, taste masking agents, buffers, antioxidants and chemical stabilizers.

The active ingredient of formula (I) may be used alone or in conjunction with other anti-inflammatory agents such as glucocorticosteroids (e.g., hydrocortisone, prednisone, prednisolone, dexamethasone, betamethasone) and non steroidal anti-inflammatory drugs (e.g., ibuprofen, naproxen, ketoprofen, diclofenac, piroxicam, celecoxib and etoricoxib).

The compound having the formula I) or its analog or pharmaceutical composition containing thereof, and optionally another anti-inflammatory agent, may be packaged and sold in the form of a kit. For example, the composition might be in the form of one or more oral dosage forms such as tablets or capsules. The kit may also contain written instructions for carrying out the inventive methods and/or the intended use of the compound or the pharmaceutical composition as described herein.

In general, treatment regimens may be designed and optimized by those skilled in the art. For example, the active may be administered until demonstrable symptoms of the inflammatory condition have substantially diminished or the condition is substantially alleviated or healed.

The term “about” refers herein to 10% more or less of the value which it refers to.

### Working Examples

The present invention will now be described in terms of the following non-limiting working examples.

### General Reagents

FTS was synthesized as previously described and was stored in chloroform, which was evaporated under a stream of nitrogen immediately before use. RFP-N-Ras and GFP-Raf-1-RBD constructs were described and validated previously.

### Cells and Transfection

The LAD2 MC, expressing functional FCεRI receptors, were established from bone marrow aspirates of a patient with MC leukemia and maintained as previously described. Transfection of LAD2 cells was performed with DMRIE-C (Invitrogen, Carlsbad, Calif.), and cells were studied 24 hours later.

### Mast Cell Stimulation and Activation

For IgE-mediated activation, cells were sensitized overnight with 100 ng/ml of human myeloma IgE-biotin (Calbiochem; Merck Darmstadt, Germany) and then stimulated with 100 ng/ml of streptavidin (Jackson Immuno-Research Laboratories, West Grove, Pa.). Degranulation was quantified 20 minutes after stimulation by assaying the release of ε-hexosaminidase, a preformed mediator present in the secretory granules of MC. Release of ε-hexosaminidase was measured spectrophotometrically by assaying the cleavage of its substrate p-nitrophenyl-N-acetyl-ε-D-glucosaminide. Degranulation was expressed as a percentage of total ε-hexosaminidase activity in the cells, obtained by lysis of the cells with 0.5% triton X-100. Supernatants obtained from MC stimulated for 3 to 6 hours were examined for the released PGD₂ and TNF-α by a commercial ELISA kits, according to the manufacturer's instructions (Prostaglandin D₂-MOX EIA Kit, Cayman Chemical Company, Ann Arbor, Mich., and TNF-α HS Elisa kit, R&D® Systems Inc., Minneapolis, Minn.).

### Ras Activation Assay

Methods of detecting activated Ras by pull-down assay and by SDS-PAGE immunoblotting were performed as previously described.

### Mice

The study was approved by the Institutional Ethics Committee of Tel Aviv University. 8-10 weeks old BALB/c female mice were primed to express an IgE-dependent passive cutaneous anaphylaxis (PCA) reaction. Mice were lightly anaesthetized with ether and their left footpads were injected subcutaneously with 20 ul (20 ng) of IgE anti DNP. Their right footpads were injected with 20 ul of diluents. The next day the mice received an intraperitoneal injection of 100 ug of DNP₃₀₋₄₀-HAS in saline. Footpad swelling was measured with an engineer's micrometer (Ozaki Mfg. Co., Itabashi, Tokyo). Footpad swelling was expressed as the difference in thickness before and after the challenge, measured at several time points after the challenge. FTS was orally administrated before the antigen challenge at 100 mg/kg as previously described.

### Microscopy

Live cells were plated in 35-mm dishes containing a no. 0 glass cover slip over a 15-mm cutout (MatTek, Ashland, Mass.). Cells were maintained at 37° C. using a PDMI-2 microincubator (Harvard Apparatus, Holliston, Mass.). Individual cells were imaged before and after addition of stimuli. Images were acquired with a Zeiss 510 inverted laser scanning confocal microscope (Carl Zeiss Microlmaging, Inc., Thornwood, N.Y.) and processed with Adobe Photoshop CS.

### Statistical Analysis

Data were analyzed and expressed as mean (±SEM). Significance was determined using Student's t-test.

### Results

### FTS Inhibits Ras Activation in Mast Cells

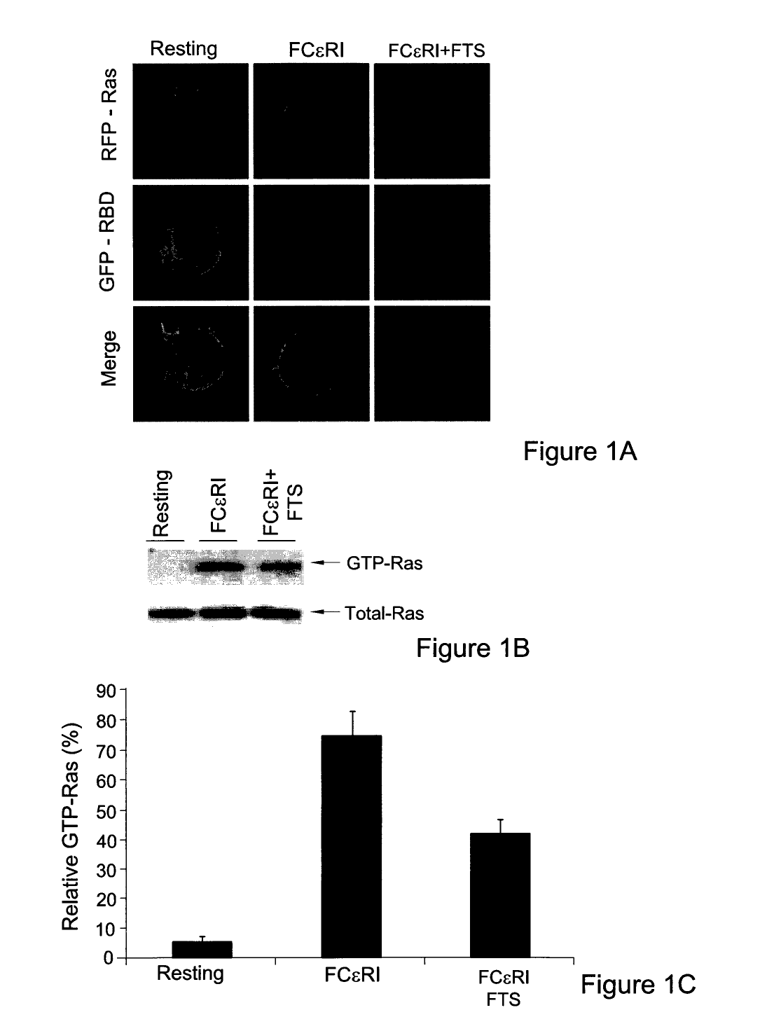

First, it was established that FTS inhibits Ras activation in MC. Ras is activated at the plasma membrane of LAD2 cells upon cross-linking the antigen receptor. LAD2 cells expressing RFP-N-Ras and GFP tagged Raf-1-RBD (marker for GTP loaded Ras) were studied (FIG. 1A). In resting cells, the probe for activated Ras was homogenously distributed throughout the cytoplasm of the cells. When the cells were stimulated by cross-linking the FCεRI, the probe translocated to the plasma membrane. This translocation was completely blocked when the cells were pretreated with 25 μM of FTS (FIG. 1A), suggesting that N-Ras activation at the plasma membrane is inhibited by FTS. In order to document the ability of FTS to inhibit the endogenous pool of Ras, LAD2 cells were subjected to Raf-1-RBD pull-down assay (FIG. 1B). Compared to serum starved resting cells, cross-linking of the FCεRI resulted in higher levels of GTP loaded Ras (FIG. 1B). When the cells where pretreated with 25 μM of FTS, the levels were significantly attenuated (FIG. 1B). Quantification of the same experiment revealed approximately 35% relative reduction in the amount of activated Ras (FIG. 1C). Thus, the results demonstrate that FTS inhibits Ras activation in MC stimulated through the FCεRI receptor.

### FTS Preferentially Inhibits Prostaglandin D₂ Generation and Release of Tumor Necrosis Factor-α

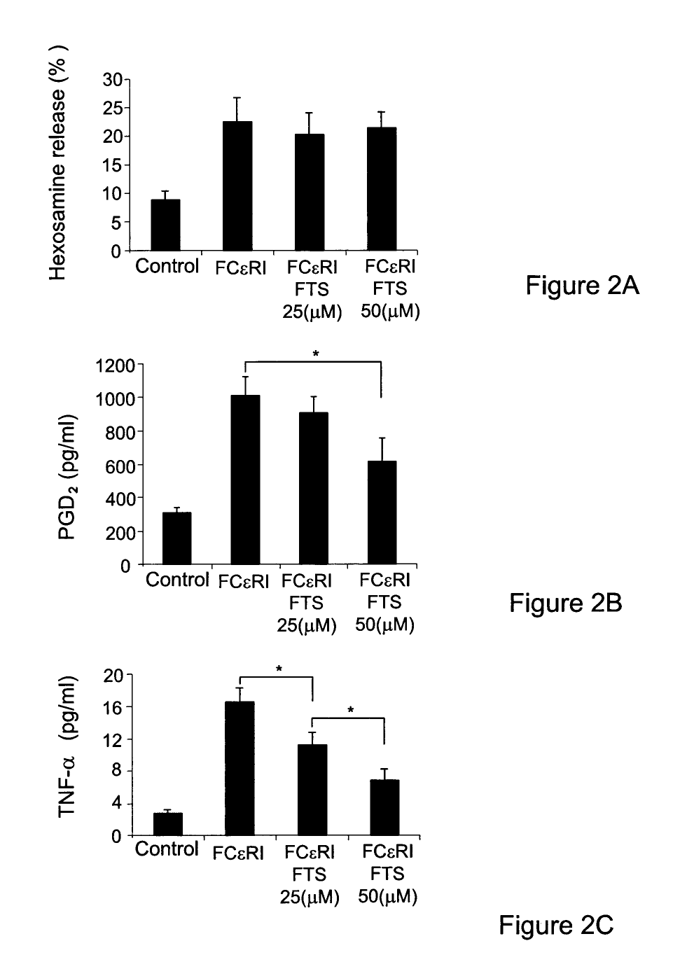

Second, the effect of FTS on MC degranulation and mediators release was investigated. LAD2 cells were activated by cross-linking the FCεRI. Degranulation, PGD₂, and TNF-α release were measured at 20 minutes, 6 and 3 hours post activation, respectively. As shown in FIG. 2A, cross-linking the FCsRI resulted in increased level of β-hexosaminidase secretion. Pre treating the cells with either 25 μM or 50 μM of FTS did not significantly modify the immunological degranulation (FIG. 2A).

In contrast, 50 μM but not 25 μM of FTS inhibited the release of PGD₂ from LAD2 cells stimulated by crosslinking the antigen receptor (FIG. 2B). Interestingly, FTS appreciably inhibited TNF-α release from LAD2 cells stimulated by cross-linking the FCεRI (FIG. 2C). Moreover, the inhibition of TNF-α release was dose dependent and statistically significant (p<0.05, FIG. 2C). Thus, FTS selectively inhibits PGD₂ and TNF-α release from activated MC without affecting their degranulation.

### In Vivo Inhibition of Passive Cutaneous Anaphylaxis Reaction by FTS

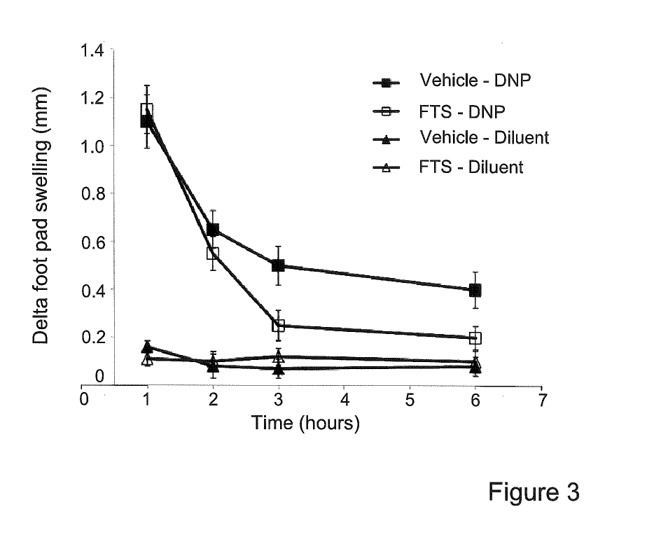

Third, it was demonstrated that FTS inhibits MC activation in an animal model. Passive cutaneous anaphylaxis reaction elicited in footpads of female BALB/c mice was studied by assessment of tissue swelling. It has been established that the increased tissue swelling is MC dependent cutaneous response, and that this reaction is regulated by MC mediators' release. Twenty (20) mice were divided into two groups. Mice of one group were treated orally with FTS (100 mg/kg), while the control group was given vehicle. The left footpads of all mice were injected subcutaneously with IgE (anti HSP), while the right footpads were injected with diluents. Footpad swelling was measured at different time points after antigen (DNP₃₀₋₄₀-HAS) challenge. As shown in FIG. 3, FTS inhibited tissue swelling as early as three hours after the challenge. The same degree on attenuation (˜40%) was observed as long as six hours into the elicitation of the allergic reaction (FIG. 3). Interestingly, the time course for FTS induced inhibition of tissue swelling in vivo correlated with the kinetic of mediators release in vitro (FIGS. 2B and 2C).

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1-21. (canceled)

22. A method of treating a mammalian subject afflicted with a non-autoimmune inflammatory condition, comprising administering to the subject a pharmaceutical composition comprising an effective amount of S-farnesylthiosalicylic acid (FTS) or a structural analog thereof, collectively defined in accordance with formula (I): 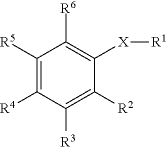
wherein X represents S; R¹ represents farnesyl, or geranyl-geranyl; R² is COOR⁷, CONR⁷R⁸, or COOCHR⁹OR¹⁰, wherein R⁷ and R⁸ are each independently hydrogen, alkyl, or alkenyl; wherein R⁹ represents H or alkyl; and wherein R¹⁰ represents alkyl; and wherein R³, R⁴, R⁵ and R⁶ are each independently hydrogen, alkyl, alkenyl, alkoxy, halo, trifluoromethyl, trifluoromethoxy, or alkylmercapto, and a pharmaceutically acceptable carrier.

23. The method according to claim 22, wherein the mammalian subject is a human.

24. The method according to claim 22, wherein the non-autoimmune inflammatory condition is an allergic inflammatory condition.

25. The method according to claim 24, wherein the allergic inflammatory condition is a pulmonary inflammatory condition.

26. The method according to claim 24, wherein the allergic inflammatory condition is asthma.

27. The method according to claim 24, wherein the allergic inflammatory condition is a gastrointestinal inflammatory condition.

28. The method according to claim 24, wherein the allergic inflammatory condition is a systemic (multi-system) allergic conditions induced by an allergen selected from the group consisting of food, drugs, latex and pets.

29. The method according to claim 22, wherein the inflammatory condition is a non-allergic inflammatory condition.

30. The method according claim 22, wherein the pharmaceutical composition comprises an effective amount of FTS.

31. The method according to claim 22, wherein FTS or its structural analog is administered orally.

32. The method according to claim 22, wherein FTS or its structural analog is administered parenterally.

33. The method according to claim 22, wherein FTS or its structural analog is administered transdermally.

34. The method according to claim 22, wherein FTS or its structural analog is administered in the form of an aerosol.

35-36. (canceled)
